SEQUENCE LISTING (17 sequences)

SEQ ID NO: 1 - protein, 16 aa, Artificial Sequence
  Description of Artificial Sequence Synthetic peptide
CLELEEELKTVTNNLK

SEQ ID NO: 2 - protein, 17 aa, Artificial Sequence
  Description of Artificial Sequence Synthetic peptide
DDEETTALVCDNGSLVK

SEQ ID NO: 3 - protein, 12 aa, Artificial Sequence
  Description of Artificial Sequence Synthetic peptide
VCDNGSLVKAGF

SEQ ID NO: 4 - protein, 22 aa, Artificial Sequence
  Description of Artificial Sequence Synthetic peptide
LCYVALDFENEMATAASSSLEK

SEQ ID NO: 5 - protein, 11 aa, Artificial Sequence
  Description of Artificial Sequence Synthetic peptide
RCPETLFQPSF

SEQ ID NO: 6 - protein, 11 aa, Artificial Sequence
  Description of Artificial Sequence Synthetic peptide
ITYGQCGDVLR

SEQ ID NO: 7 - protein, 14 aa, Artificial Sequence
  Description of Artificial Sequence Synthetic peptide
TECFVPDDKEEYVK

SEQ ID NO: 8 - protein, 15 aa, Artificial Sequence
  Description of Artificial Sequence Synthetic peptide
DIRTECFVPDDKEEY

SEQ ID NO: 9 - protein, 10 aa, Artificial Sequence
  Description of Artificial Sequence Synthetic peptide
ADAEERCDQL

SEQ ID NO: 10 - protein, 20 aa, Artificial Sequence
  Description of Artificial Sequence Synthetic peptide
MDADLSQLQTEVEEAVQECR

SEQ ID NO: 11 - protein, 10 aa, Artificial Sequence
  Description of Artificial Sequence Synthetic peptide
KLEDECELKR

SEQ ID NO: 12 - protein, 13 aa, Artificial Sequence
  Description of Artificial Sequence Synthetic peptide
ELPPDQAQYCIKR

SEQ ID NO: 13 - protein, 15 aa, Artificial Sequence
  Description of Artificial Sequence Synthetic peptide
VEFECEVSEEGAQVK

SEQ ID NO: 14 - protein, 13 aa, Artificial Sequence
  Description of Artificial Sequence Synthetic peptide
ECEVSEEGAQVKW

SEQ ID NO: 15 - protein, 18 aa, Artificial Sequence
  Description of Artificial Sequence Synthetic peptide
AAFDIFVLGAEDGCISTK

SEQ ID NO: 16 - protein, 29 aa, Rattus sp.
LFDRTPKGEMKITYGQCGDVLRALGQNPT

SEQ ID NO: 17 - protein, 27 aa, Rattus sp.
LFDRTGECKITLSQVGDVLRALGTNPT

(19) United States
(12) Patent Application Publication
(10) Pub. No.: US 2012/0231482 A1
(43) Pub. Date: Sep. 13, 2012

(54) METHOD, KIT OR DIAGNOSTIC FOR THE DETECTION OF REAGENTS WHICH INDUCE ALTERED CONTRACTILITY

(75) Inventors: Christopher I. Murray, Baltimore, MD (US); Jennifer E. Van Eyk, Baltimore, MD (US); Nazareno Paolocci, Baltimore, MD (US); Wei Dong Gao, Woodstock, MD (US); Darren Brian Foster, Baltimore, MD (US)

(73) Assignee: THE JOHNS HOPKINS UNIVERSITY, Baltimore, MD (US)

(21) Appl. No.: 13/509,397

(22) PCT Filed: Nov. 15, 2010
(86) PCT No.: PCT/US10/56736

§ 371 Date: May 11, 2012

Related U.S. Application Data
(60) Provisional application No. 61/260,888, filed on Nov. 13, 2009.

(51) Int. Cl. C12Q 1/02; C07K 14/47; C12Q 1/37; G01N 33/53; G01N 27/62

(52) U.S. Cl. 435/7.92; 435/29; 435/7.1; 435/23; 530/350

(57) ABSTRACT

A method of screening for compounds that enhance or depress contractile function, based on measuring the formation of heterodimers of contractile fibers (e.g. Tm and actin, myosin heavy and myosin light chains), for example through disulfide bond formation. Diagnostic and prognostic methods and kits are also provided.

### BACKGROUND

Contraction in muscle and other contractile cells occurs through the processive binding and release of myosin heads to actin filaments causing the thick and thin filaments to slide past one another shortening their combined effective length. The thick filament is comprised of myosin (consisting of myosin heavy (MHC) and 2 different myosin light chains (in cardiac muscle myosin light chain 1 (MLC1 also called MLC3) and myosin light 2) and myosin binding protein C while the thin filament is comprised of filamentous actin, tropomyosin (TM), and in case of skeletal and cardiac muscle the troponin (Tn) complex (comprising troponin I (TnI), troponin T (TnT) and troponin C (TnC) while in smooth muscle and other contractile cells, caldesmon and calponin among others. The alignment of the myofilament requires additional proteins such as alpha-actinin. For striated muscle (cardiac and skeletal) regulation of this process is achieved through the precise and adjustable arrangement of the myofilament accessory proteins; TM the troponins (TnI, TnT, and TnC) and myosin light chains one and two. Contraction is initiated by the binding of calcium to TnC causing a conformational change in the structure of the troponin complex allowing tropomyosin to move from its B-state (blocked) position over the myosin binding sites on the actin filament to a C-state (closed) position. This movement allows myosin heads to attach further displacing tropomyosin to the M-state. With sufficient calcium concentration, cooperative binding events occur along the length of the thin filament activating it, allowing for repeated myosin interactions producing a contractile event [1].

In disease, a cell's ability to handle calcium can become diminished either through impaired calcium cycling for each contractile event or a myofilament insensitivity to the available cellular calcium. In either case, contractile efficiency is reduced usually resulting in a pathology and determent to the individual. There is considerable interest in the development of interventions that can improve or reverse these effects to restore healthy function [2, 3]. One class of compounds that holds promise to improve function in failing hearts by increasing inotropy via enhanced sensitization of myofilaments to Ca²⁺ are nitroxyl (HNO) donors [4, 5]. HNO is a nitric oxide (NO.) which is known to react with free thiol groups in proteins, forming either sulfinamide or disulfide bonds. The present inventors' investigations into these thiol reactive compounds have lead to the discovery of two previously uncharacterized modifications and novel mechanisms for increased maximum contractile force and increased calcium sensitivity.

Contractile cells and tissues including the heart, skeletal and smooth muscle (and other mobile cell types) can have reduced or increased contractility or mobility with disease or treatment with drugs. There is a need for reagents which can modulate contractility. The present inventors have determined that direct crosslinking resulting in the formation of heterodimers primarily of tropomyosin (TM) and actin as well as of myosin heavy chain and myosin light chain 1 produces a positive inotropic effect enhancing contraction. Other disulfide bonds can simultaneously occur including within actin and MHC and between actinin, myosin binding protein C and Troponin C. As such, any bioactive agent capable of inducing such a covalent crosslink would be able to confer this effect on contraction. In contrast, an agent capable of inhibiting such covalent crosslinking would be expected to reduce contractility. Such agents should be useful in the diagnosis and treatment of a variety of conditions that involve contractile cells. Biomarkers which can detect the formation of specific disulfide bonds can reflect the contractility of the heart as well as the effectiveness of the reagents that induce or inhibit the formation of these disulfide. This would allow monitoring of the reagent inducing disulfide formation.

### SUMMARY

The present inventors have discovered that the formation of a covalent crosslink (eg. Asp-Lys side chain or cysteine disulfide bond) between TM-actin and or myosin heavy chain and light chain 1 confers positive inotropic effect in contractile cells and tissues. In accordance with this finding, a reagent that is able to induce or inhibit the formation of this modification should modulate contractility. Other disulfide bonds that occur to the myofilament proteins at the same time as these functional disulfides could also be used to monitor the underlying biological effect. The present application describes a method, kit or diagnostic for the detection of reagents which can alter contractility through the formation of these disulfide bond heterodimers.

In one aspect, the invention provides a system or method for the screening for compounds that improve or depress contractile function based on this crosslinking. The disulfide bond formation (e.g dimer formation) can be monitored at level of muscle or contractile tissue, isolated cells, isolated myofibrils, isolated proteins or peptides of the regions where the crosslinking occurs. Detection of the crosslinking can be demonstrated by formation of the covalent crosslink, antibodies or other detecting reagents, molecular weight alterations, and mass spectrometry based methods.

Compounds identified by the aforementioned method can be tested for in vivo efficacy and, if suitable, used in treating diseases or disorders that require increasing or decreasing contractility in a variety of tissues and cells, including, but not limited to cardiac, skeletal and smooth muscle, blood cells such as neutrophils and platelets, and cancer cells, e.g. metastatic cells having motility or potential motility.

Diseases or disorders that can be treated using such agents include, but are not limited to, those involving any dysfunctional contractile tissue including, but not limited to heart failure, heart contracture induced by cardiac injury, skeletal muscle cramps, irritable bowl and gastric mobility, Crohn's disease, asthma, vascular spasm, uterine contraction involved in premature delivery or delivery itself, menstrual cramps, atrophy due to muscle wasting (bed rest or being on a ventilator) and deficiencies, as well as hyper-contractile conditions if the underlying cause is related to the establishment of the one or both of the described crosslinks. Motile cells (such as smooth muscle and neutrophils, lymphocytes) the actin-TM crosslink alone may be sufficient to confer altered motility. Induction of such crosslink would increase motility for example smooth migration to the vascular lumen upon injury.

The invention also includes a diagnostic to be used for the detection of the heterodimers resulting from the crosslinking described above. Such diagnostics can be used, e.g., for monitoring treatment responsiveness and outcomes, adjusting dosages, etc., when agents identified by the invention and other known agents are used in treatment of a disease or disorder (for example those mentioned above).

In another aspect, the invention provides a kit for use in carrying out the aforementioned method. In one embodiment, the kit comprises, e.g., reagents and components needed for contacting contractile proteins with test agents and measuring heterodimer formation and/or an increase in heterodimer level. Thus, the kit may include materials needed to carry out separation of products such as nitrocellulose membranes, detection agents such as primary antibodies against specific homo or heteromer dimers or intramolecular crosslinked molecules and/or heterodimers (e.g. antibody to tropomyosin, antibody to actin), controls such as homodimers (e.g. tropomyosin homodimer, myosin light chain 1, myosin heavy chain α/β), ELISA plate coated with tropomyosin detection antibody, antibodies against the synthetic peptides of Table 1, digestive enzymes such as trypsin and chymotrypsin, buffers, etc. The kit will be required to distinguish the crosslink directly (via an antibody or change in molecular weight) or indirectly when the endogenous target Cys is chemically modified and the antibody is against the specific induced Cys-. For example Cys involved in the disulfide could be selectively reduced and subsequently specifically modified with a chemical moiety to which an antibody has been produced to recognize.

In another embodiment, the kit comprises, e.g. reagents and components needed for measuring a decrease in the level of heterodimers when an agent is added. This kit may, in addition to the above, include, e.g., at least one reagent to stimulate formation of crosslinking between contractile fibers, so that test agents can be added to assay for their ability to inhibit or reverse such crosslinking.

This invention is based on work that has induced, characterized and identified this crosslinking phenomenon and in doing so developed a “diagnostic” assay for its detection allowing evaluation of any potential bioactive compound for ultimate use in manipulating contraction. This includes all contractile tissues such as cardiac, smooth and skeletal muscle as well as motile cells such as neutrophils. Thus, the term “contractile cell” or “contractile tissue” is intended to include cardiac, smooth and skeletal muscle, motile blood cells such a neutrophils, platelets and lymphocytes, cancer cells, fibroblasts, stem cells, endothelial cells, and epithelial cells and any cell that contains having contractile proteins or stress fiber proteins that comprise of actin-TM and or myosin

The present inventors have identified a cross link (in this case, a disulphide bond but any covalent crosslink would be applicable) between TM and actin and/or myosin heavy and myosin light chains that alters the arrangement of the thin and thick filament reducing the requirement for calcium to achieve activation and resulting contraction. These findings represent a new modification of contractile proteins, specifically TM, actin and myosin heavy and light chain 1 and confer a novel mechanism for effecting contractile response. For example, the disulfide bonds between Cysteine residues 190 of TM and Cys 257 of actin, and/or the disulfide bond between Cys 37 of myosin heavy chain and Cys 81 of myosin light chain 1, is sufficient to cause inotropic action. Other disulfide bonds that can occur simultaneously can also be used as diagnostic including actin to actin, MHC to MHC and between actinin, myosin binding protein C and Troponin C. The quantity of crosslinking can be measure (e.g. based on MW (such as one dimensional SDS-PAGE and size exclusion chromatography) with detection of (e.g western blot using Ab against protein or the modification or mass spectrometry) the ternary structure of TM, actin, myosin heavy chain and myosin light chain 1. For the TM-actin interaction, higher molecular weight complexes intermediate in size compared to a TM homodimer (smaller) and the actin homodimer (larger) indicate the diagnostic heterodimeric form. In the case of the myosin interaction, a higher molecular weight form of myosin light chain 1 can be observed at 230 kDa (the approximate weight of myosin heavy chain plus light chain 1). The sensitivity to selective reducing agents can be used as control. The direct detection of the crosslink can also be done in which MS or antibody is used. Mimmetics of the cross bridge such as Asp-Lys covalent bond or the selective labeling (switch) of the disulfide with a chemical moiety which can be detected by a specific antibody or mass spectrometry with or with pre-enrichment of the protein complexes.

The formation and quantification of either or both of these homo and heterodimers by any means is indicative of the myofilament (thick and thin filament) structural alteration that is necessary to retune the apparatus and confer the increased contractile performance. Conversely, this invention can be used for the evaluation of compounds that prevent or reverse the formation of the TM-actin heterodimer. This type of intervention would also be valuable in addressing hypercontractile conditions, such as skeletal or smooth muscle cramping. The dimers can be monitored at level of muscle or contractile tissue, isolated cells, isolated myofilbrils, isolated proteins or peptides of the regions where the crosslinking occurs. Formation of the crosslinking can be detected by the formation of a covalent crosslink, antibodies or other detecting reagents, MW alterations, MS based methods. Detection alone could be used as a biomarker (tissue or cell based or body fluid such as serum or plasma for molecular assessment of contractility and whether the therapeutic would be useful on a patient to patient bases.

### BRIEF DESCRIPTION OF THE DRAWINGS

FIG. 1. Effect of NCA on force and intracellular Ca²⁺ transient in rat cardiac muscle. (A) Raw tracings of intracellular Ca²⁺ transient (left) and force (right) at varied NCA concentrations. (B) Pooled data of the dose-response of [Ca²⁺]ᵢ (left) and force development (right) to NCA (0-200 mmol/L). Note that systolic force increased significantly without increases in diastolic force at varied NCA concentrations. N=7-8 in each group (C) Effect of NCA on systolic [Ca²⁺]ᵢ transient (left) and force development (right) at varied external Ca²⁺. At any given [Ca²⁺]ₒ, systolic force increased significantly after NCA while systolic [Ca²⁺]ᵢ transient was not affected. * p<0.05 vs. no drug, n=5 in each group. (D) Effect of NCA on force-frequency relation. NCA did not affect Ca²⁺ transient at any given frequencies of stimulation but increased force development at higher stimulation frequencies. * p<0.05 vs. no drug, ** p<0.01 vs. no drug, n=6 in each group.

FIG. 2. Effect of NCA on steady-state force-[Ca²⁺] relationship in cardiac muscle. (A) Steady-state force-[Ca²⁺]ᵢ relationship in intact trabeculae before and after NCA (20 μmol/L). n=5. See text for details. (B) Force-[Ca²⁺] relation in skinned trabeculae before and after NCA (n=6). (C) Reversal of NCA's effect on force-[Ca²⁺] in skinned muscles. The muscles were treated with DTT (5 mM) for 10 min after 1^{st} force-[Ca²⁺] was obtained in the presence of NCA alone, and a second force-[Ca²⁺] relation was obtained in the presence of NCA+DTT. n=3. (D) For comparison, steady-state force-[Ca²⁺]ᵢ relationship in intact trabeculae before and after AS (500 μmol/L). n=5.

FIG. 3. Detection and capture of HNO modified myofilament proteins. (A) Modified biotin switch assay schema outlining thiol blocking, HNO reversal and biotin labeling steps as well as capture of intact proteins or digested proteins for MS/MS mapping of sites of HNO modification. (B) silver stained gel of rat cardiac myofibrils treated with HNO/NO donors or control compounds, subjected to the biotin switch assay and eluted from streptavidin agarose. Of note, HNO modifications were reduced by 5 mM DTT but were resistant to treatment with 1 mM ascorbate (black arrowheads) while NO modifications were reversed with ascorbate (outline arrowhead). (C) Experimental strategy for the assignment of candidate modified cysteine residues identified by site mapping study. Modifications in common between NCA and AS treatments were considered to be candidates for the increase in max force while modifications specific to NCA treatment were likely responsible for the decrease n Ca₅₀.

FIG. 4. Validation and characterizations of HNO modifications. 1 μg of rat cardiac myofibrils treated with HNO donors or control compounds were separated under reducing or nonreducing conditions and western blots probed for candidate proteins tropomyosin, actin, myosin heavy chain, myosin light chain 1 (n=4). In each case a change in mobility was observed for HNO treated samples which was reversed with treatment of 5 mM DTT. In addition, a loss of epitope was observed for actin with NCA treatment (lane 4). Silver stained gel analysis indicated a protein band with increased gel mobility that was lost with DTT. MS analysis confirmed this band to be actin and also revealed a similar, but less abundant, shift for actin in AS treated samples (n=2).

FIG. 5. Evaluation of the interaction between actin and tropomyosin with NCA treatment. Purified rabbit skeletal tropomyosin (0.03 μg) and isolated rat cardiac myofibrils (1 μg) were treated with NCA or AS and evaluated by 1D non-reducing western blot probing for tropomyosin (A) and actin (B) (n=3). Analysis revealed that treatment of myofibrils with NCA produced a higher molecular weight form of both tropomyosin and actin that were specific to NCA treatment. (C) Fluorescent DIGE analysis of samples from A and B independent labeled indicates NCA specific band (green) above purified TM (blue) and below AS treated myofibrils (red) (n=3). (D) MS analysis of the same gel region identified both tropomyosin and actin in non-reduced lane but not with DTT treatment (n=2).

FIG. 6. MLC1 Cys81 necessary for increase in Fₘₐₓ induced by treatment with HNO donors. (a) Sequence alignment comparing isoforms of rat cardiac (SEQ ID NO:16) and skeletal MLC1 (SEQ ID NO:17) in the region surrounding cardiac Cys81. (b) Steady-state force-[Ca²⁺]ᵢ relations in control (o) versus NCA-treated (•) from cardiac or skeletal trabeculae indicating loss of increase in maximum force with loss of Cys81 in skeletal isoform with NCA (n=5, each group). (c) 2D Gel shift assay (non-reduced, NR and reduced, DTT) using 10 μg of skeletal or cardiac myofibrils indicating loss of higher molecular weight forms of MLC1 in skeletal HNO treated preparations while maintaining higher molecular weight form of TM (n=3).

FIG. 7. Proposed mechanism of NCA induced increase in Ca²⁺ sensitivity. Depicted on the left is a representation of the thin filament with the relative positions of tropomyosin in the relaxed (B-state—black) and Ca2+ activated state (C-state—blue). Subdomains of actin (1-4) relative to tropomyosin position are indicated in the blow up (light grey) along with the approximate location of the weak (green) and strong (red) myosin binding sites. HNO induced crosslinking of tropomyosin (Cys190) to the inner domain of actin (subdomain 4, Cys 257) imposes restraints on tropomyosin movement that bias its equilibrium position toward the Ca²⁺-activated state (blue). This effect increases the availability of some myosin binding sites in the relaxed or B-state lowering the threshold for Ca²⁺ activation providing a mechanism for HNO enhanced myofilament response to Ca²⁺.

FIG. 8. Proposed mechanism and validation of pinned myosin head hypothesis of increase in maximum force generation. (A) diagram of thin and thick filament including position of myosin light chain 1 relative to head region of heavy chain indicating the effect a cross link between two proteins could have.

### DETAILED DESCRIPTION

In accordance with the invention, methods, diagnostics and kits are disclosed herein that relate to alterations in the properties of contractile cells and tissues, such as, for example, heart, skeletal and smooth muscle, motile blood cells such as neutrophils, and cancer cells.

In one aspect, the invention includes a method for screening for an agent that increases contractility in a contractile cell comprising the steps of

a) contacting a test agent with a composition comprising contractile proteins from said cell; and
b) measuring the formation of at least one cross link between said contractile proteins;
wherein the formation of at least one cross link between said proteins is indicative of an potential agent for increasing contractility of said cell.
In one embodiment, measurement of a cross link is accomplished by assaying for the presence of, or an increase in, heterodimers formed by the contractile proteins.

The contractile cell can be, for example, a muscle cell, e.g., a smooth muscle cell, a skeletal muscle cell or a cardiac muscle cell. The contractile cell may also be a cell having motility, such as a blood cell (a neutrophil or lymphocyte), or a cancer cell, in particular a metastatic cancer cell.

The contractile proteins may be, e.g., TM and actin, myosin heavy and myosin light chains alone or in combination. The cross link can be, for example, a disulfide bond, or any other covalent bond that is induced by the test agent.

In specific embodiments, the cross link is formed between Cysteine residue 190 of TM and Cys 257 of actin, or between Cys 37 of myosin heavy chain and Cys 81 of myosin light chain 1.

Cross-linkage can be measured by any suitable method known in the art, for example, by molecular weight assay, antibody assay, molecular sieving assay, mass spectrometry as well as using a switch assay which the non-modified Cys are blocked and the disulfide bond is reduced. The Cys that were involved in the disulfide bond are then chemically modified. In this case the modification is detected by antibody raised against the chemical moiety or directly by mass spectrometry. In the latter case, quantitation can be carried out if the moiety has different molecular weights. Then when different samples are labeled with the moiety each with a different molecular weight, the ratio can be determined between samples. Alternatively, a recombinant or synthetic protein or peptide containing the modified Cys is produced which has a different mass than the endogenous form. Spiking in a known amount of the labeled form allows quantification with respect to the endogenous Cys.

Also provided is a method for screening for an agent that reduces contractility in a contractile cell comprising the steps of

a) contacting a test agent with a composition comprising contractile proteins from said cell in homo or heterodimer form; and
b) measuring the disruption of at least one cross link between said contractile proteins;
wherein the disruption of at least one cross link between said proteins is indicative of a potential agent for decreasing contractility of said cell.

The contractile cell can be, for example a muscle cell, e.g., a smooth muscle cell, a skeletal muscle cell or a cardiac muscle cell. The contractile cell may also be a cell having motility, such as a blood cell (a neutrophil or lymphocyte), fibroblast, stem cell, endothelial or epithelial or a cancer cell, in particular a metastatic cancer cell or any cells that can be motile and contains actin, TM and or myosin.

The contractile proteins may be, e.g., TM and actin, myosin heavy and myosin light chains, actinin, myosin binding protein C and Troponin C. The cross link can be, for example, a disulfide bond, or any other covalent bond that is induced by the test agent.

In specific embodiments, the cross link is formed between Cysteine residue 190 of TM and Cys 257 of actin, or between Cys 37 of myosin heavy chain and Cys 81 of myosin light chain 1.

Cross-linkage can be measured by any suitable method known in the art, for example, by molecular weight assay, antibody assay, molecular sieving assay, mass spectrometry. As well as using a switch assay which the non-modified Cys are blocked and the disulfide bond is reduced. The Cys that were involved in the disulfide bond are then chemically modified. In this case the modification is detected by antibody raised against the chemical moiety or directly by mass spectrometry. In the latter case, quantitation can be carried out if the moiety has different molecular weights. Then when different samples are labeled with the moiety each with a different molecular weight, the ratio can be determined between samples. Alternatively, a recombinant or synthetic protein or peptide containing the modified Cys is produced which has a different mass than the endogenous form. Spiking in a known amount of the labeled form allows quantification with respect to the endogenous Cys.

Agents or compounds identified by the methods above can be screened further for use in the treatment, diagnosis, and prognosis of a variety of disorders and diseases involving contractile cells, as detailed elsewhere herein.

Also provided is a diagnostic method comprising the step of detecting and/or measuring the level of a heterodimer comprised of contractile proteins in a biological sample wherein the presence and/or level of said heterodimer is correlated with a diagnosis, prognosis or treatment outcome.

Diseases, disorders and dysfunctions to be diagnosed, treated, monitored or prognosticated include cardiac disorders such as heart failure and myocardial stunning, diseases/disorders/dysfunctions of skeletal muscle such as skeletal muscle cramps, hypercontraction, diseases/disorders/dysfunctions of smooth muscles such as irritable bowl and gastric mobility, asthma, vascular spasm, uterine contraction involved in premature delivery or delivery itself, menstrual cramps, cancer, in particular metastatic cancer

The biological sample can be a blood sample (e.g. whole blood, serum or plasma) or other tissue sample, such as a sample obtained by tissue biopsy (e.g. a cardiac, skeletal or smooth muscle biopsy) or during surgery, or a bodily fluid such as urine, saliva, sweat, etc.

Also provided are kits containing compositions and reagents for practicing the invention.

In one embodiment, the kit contains reagents, etc., for carrying out a Gel based method. One such kit, for detecting actin-TM dimer by WB may comprise, for example, one or more of the following:
Bis-tris gel(or any denaturing gel type including SDS PAGE), MOPS running buffer
Nitrocellulose membrane
Detector: primary antibodies, e.g., antibody to tropomyosin (e.g. CH1, Sigma-Aldrich Co., St. Louis, Mo., USA), antibody to actin (e.g. AC-40, Sigma-Aldrich Co., St. Louis, Mo., USA)
MS, MRM, aptimeter, etc.

Alternative proteins could be detected by MS, MRM, aptimeter, etc.
Control: tropomyosin homodimer as molecular weight standard

Another such kit, for detecting myosin heavy chain (MHC)-myosin light chain (MLC1) dimers, may comprise one or more of the following:
enrichment: bis-tris gel (or any denaturing gel type including SDSPAGE), MES running buffer
Nitrocellulose membrane
Detectors: primary antibodies, e.g., myosin light chain 1 (e.g. MLM527, Abcam, Cambridge, Mass., USA), myosin heavy chain α/β (e.g. 3-48, Abcam, Cambridge, Mass., USA).
   Alternative proteins could be detected by MS, MRM, aptimeter, etc.

In another embodiment, the kit contains reagents, etc., for detecting heterodimers by ELISA assay.

One such kit, for indirect detection of actin and TM dimers, may comprise, for example, one of more of the following:
enrichment: Spin column with 50 kDa MW cut off or any size exclusion column system that differentiates between 32 and 70 kDa.
detector: ELISA plate coated with tropomyosin (e.g.CH1, Sigma-Aldrich Co., St. Louis, Mo., USA)
detection antibody: actin (e.g. AC-40, Sigma-Aldrich Co., St. Louis, Mo., USA) conjugated with a fluorophore.
wash buffer
controls:
   negative control: reducing buffer (DTT solution)
   positive control: synthetic peptide containing both antibody epitopes

Another such kit, for direct detecting actin and TM dimer or myosin heavy chain (MHC)-myosin light chain) MLC1 dimers, may comprise, for example, one or more reagents needed to carry out the following protocol: One kit would contain at least an enrichment method base on MW (size exclusion filter) followed by selection of high MW fraction which is analyzed by an ELISA against actin-TM.

### Production of Antibodies Recognizing Disulfide Bond for Both Dimer Forms:

Antibodies are produced against the synthetic peptides (8-20 amino acid residues) around the Cys in the various proteins.

Oxidation of the actin and TM or myosin heavy chain and light chain is allowed. The unoxidized and the oxidize antigen are used directly or conjugated to a carrier prior to production of polyclonal and monoclonal antibodies. An alternative is generation of peptoides or aptimers are used instead of antibodies. Selection of antibodies occurs using the oxidized and unoxidized peptides in a sequential purification protocol.

Alternative: Cys can be replaced by another amino acid residue (e.g. Ala) to ensure no oxidation. As well, Cys can be replaced in on protein with a Lys and Asp in the other protein involved in the dimer. The crosslink between Lys and Asp can be induced using transglutaminase or chemical based methods. Selection of antibodies occurs using the oxidized and unoxidized peptides in a sequential purification protocol. An alternative is peptoides or aptimers are generated and used instead of antibodies.

### ELISA Production for Actin-TM Dimer

Solublization of tissue or isolated myofilament or individual proteins (eg. actin or TM).

Capture of either actin or TM or any protein if the myofilament is in the native form. Detection using the anti-disulfide actin-TM antibody. Control is the non disulfide bond antibody. Proteins could be detected by MS, MRM, aptimeter, etc. An alternative is to capture using the anti-disulfide actin-TM antibody with detection using either anti-nondisulfide actin or TM antibodies (or any actin and TM Ab).

### ELISA Production for Myosin Heavy Chain—Light Chain Disulfide

Solublization of tissue or isolated myofilament or individual proteins (e.g. intact myosin) Capture of myosin and detection using the anti-disulfide MHC-MLC1 antibody. Control is the non disulfide bond MHC-MLC1 antibody. Proteins could be detected by MS, MRM, aptimeter, etc. Alternative is to capture using the anti-disulfide MHC-MLC1 antibody with detection using either anti-nondisulfide MHC-MLC1 antibodies (or any MHC or MLC1 Ab.

In another embodiment, the kit comprises reagents, etc. for use in detection of heterodimers directly by mass spectroscopy.

One such kit would contain one or more reagents, etc. for carrying out digestion of tissue, cell, any body fluid (e.g. serum), isolated myofilament or isolated individual proteins (e.g. actin or TM or intact myosin or MHC or MCL1). The digestion can be carried out using chemical or enzymatic methods (e.g. a mixture of trypsin and chymotrypsin, see Appendix. Samples can be fractionated (e.g. size exclusion, etc), if needed. The digests are analyzed by mass spectrometry using MALDI TOF, MALID TOF TOF, MALDI TOF TOF TOF, and a number of different electrosparay ionization MS instruments (ESI instrumentation) including LTQ Orbitrap or Triple quadrupole mass spectrometers, MS base method can be direct observation or targeted using multiple reaction monitoring (MRM or SRM) methods. Example of MRM peptides and their transitions are in the Appendix. Quantification can be achieved by addition of a known amount of a labeled peptide (e.g. N15), peptide comprising random sequence, labeled protein (e.g. N15) etc. The minimum this kit would require is labeled peptide suitable for mass spectrometry). Most often there would be an enrichment step based on MW or immuno-precipitation of the modified proteins.

In another embodiment, the kit comprises reagents, etc. for use in detection of heterodimers directly by biotin switch capture.

One such kit contains reagents, etc. for blocking all free Cys residues in a tissue, cell, any body fluid (e.g. serum), isolated myofilament or isolated individual proteins (e.g. actin or TM or intact myosin or MHC or MCL1). Blocking buffer can consist of HEPES, NEM and SDS or other detergents). The Cys residues involved in disulfide bonds are reduced with DTT and labeled. The labeling buffer can consist of HEPES, Biotin-HPDP and SDS or other detergents. Other Cys labeling reagents can be used such as tandem mass tags that react to Cys. Analysis and detection of the modified form can be done directly at the protein or peptide level. Otherwise, the protein or peptide can be enriched and isolated using streptavidin agarose or antibody to the protein. The modified protein can be assessed directly. Otherwise, the enriched sample can be digested using chemical or an enzyme(s) and the peptides isolated using streptavidin agarose or other affinity purification methods prior to MS analysis. MS analysis can be done directly on the peptide mixture. An alternative is to target the modified peptide directly using MRM. The minimum kit would require labeling moiety and antibody against the labeling moiety or a synthetic labeled peptide with the labeling moiety attached. Most often there would be an enrichment step based on MW or immunopreciptication of the modified proteins.

In another embodiment, the kit comprises one or more components or reagents for an alternative immune assay against modified Cys proteins:

Antibody production: The Cys containing peptide (are as described above consisting of the amino acid residues around the Cys involved in the disulfide bonds of actin, TM, MHC and MLC1 (as described above)) are reacted with a Cys reactive group (e.g. Biotin-HPDP. These modified peptides (with or without a carrier group) are used as the immunogen. Either polyclonal or monoclonal antibodies (an alternative is peptoides or aptimers) are generated. The resulting antibodies (peptiodes or aptimers) are purified against the Cys modified peptides and or Cys modified proteins (Same modification as the immunogen). The antibodies may also need to be cleared against the unmodified forms of either the peptide or the proteins.
Sample preparation: The sample containing the disulfide actin-TM and or MHC-MLC1 have all free Cys residues blocked using a blocking group, like NEM which is not the same modification that is used to create the antibody. The sample containing the actin-TM and or MHC-MLC1 disulfide bonds are then reduced with DTT and modified using the same Cys reactive reagent used to generate the antibody (e.g. biotin-HPDP). This can be done in modified tissue, cells, body fluid, or isolated myofilament. One or more of the anti-peptide antibodies generated can be used directly.
ELISA: The anti-Cys modified peptide antibodies (one or more) can be used directly in a manner similar to immunohistochemistry, dot blot or gel electrophoresis on the Cys modified tissue, body fluid, cells or isolated myofilaments. Alternatively, a sandwich ELISA can be made in which the modified actin, TM, MHC or MLC1 is captured using antibody against the protein and then probed for the modification using the anti-modified peptide antibody. The minimum kit would contain the antibody against the modified protein or peptide containing the disulfide (or Asp-Lys) bond. Most often there would be an enrichment step based on MW or immunopreciptication of the modified proteins.

### DEFINITIONS

The following definitions and abbreviations have been used throughout:

### AS: Angeli's Salt

MS: Mass spectroscopy
MW: Molecular weight
ELISA: Enzyme-linked immunosorbent assay
NCA: 1-nitrosocyclohexylacetate
WB: Western blot
MRM: multiple reaction monitoring
Contractile cell: a cell comprising contractile fibers, such as a smooth, skeletal or cardiac muscle cell, a motile blood cell or one having contractile properties such as a neutrophil, lymphocyte, or platelet
Contractility: a shortening of contractile fibers. As used herein, an increase in contractility is a measurable shortening of e.g. muscle fibers, or an increase in force generated by the fibers.
Test agent: a compound or composition to be screened using the methods disclosed herein.
Cross link: a covalent bond between two contractile proteins, for example a disulfide bond.
Isolated: separated from components with which it is found naturally, but not necessarily purified to a particular level.

### Methods

The following methods were used in the examples detailed below:

Force and [Ca²⁺]ᵢ Measurements in Cardiac Trabeculae.

Rat hearts were exposed via midsternotomy after the animals were anesthetized with intraperitoneal injection of pentobarbital (100 mg/kg), and were then rapidly excised and aorta cannulated. The hearts were perfused retrogradely (˜15 ml/min) with high K⁺ Krebs-Henseleit (H-K) solution equilibrated with 95% O₂ and 5% CO₂. Trabeculae were quickly dissected from the right ventricles of the hearts and mounted between a force transducer and a motor arm. The muscles were superfused with K-H solution at a rate of ˜10 ml/min and stimulated at 0.5 Hz. Force was measured using a force transducer system (SI, Germany) and expressed in mN/mm². Sarcomere length was measured by laser diffraction [6]. Fura-2 potassium salt was microinjected iontophoretically into one cell and allowed to spread throughout the whole muscle (via gap junctions). The epifluorescence of fura-2 was measured by exciting at 380 and 340 nm. The fluorescent light was collected at 510 nm by a photomultiplier tube (R1527, Hamamatsu). [Ca²⁺]ᵢ was given by (after subtraction of the autofluorescence): [Ca²⁺]ᵢ=K′d(R-Rmin)/(Rmax-R), where R is the observed ratio of fluorescence (340/380), K′d is the apparent dissociation constant, Rmax is the ratio of 340 nm/380 nm at saturating [Ca²⁺], and Rmin is the ratio of 340 nm/380 nm at zero [Ca²⁺]. The values of K′d, Rmax, and Rmin were determined by in vivo calibrations [6]. Tetanization of the trabecula was achieved by addition of ryanodine (1.0 μmol/L) and by increasing the stimulus rate to 10 Hz briefly (˜3 sec) to obtain steady-state force-[Ca²⁺] relations. Different levels of tetanized force will be obtained by increasing [Ca²⁺] in the perfusate (up to 20-25 mmol/L). The data will be fitted with the Hill equation: F=Fmax[Ca²⁺]ᵢₙ/(K1/2n+[Ca²⁺]ᵢₙ), where Fmax is the maximal force. K1/2 is [Ca²⁺]ᵢ at half Fmax, and n is the Hill coefficient.

Modified biotin switch and Western immunoblotting. Isolation of Myofibrils

Rat myofibrillar preparations, as described in [7], were obtained from frozen ventricles (Pel Freez Biologicals) minced in 20 volumes/tissue weight of 4° C. relax buffer (SRB (75 mM KCL, 10 mM imidazole pH 7.2, 2 mM MgCl₂) plus 4 mM phosphocreatine, 1 mM ATP, 50 mM BDM, 1 mM benzamidine-HCL, 0.1 mM PMSF, 1 μg/ml leupeptin, 1 μg/ml pepstatin, 1% (v/v) Trixon X-100) and adjusted to 10 mM EDTA. Minced preparations were centrifuged for 8 min at 3000xg and the supernant was decanted. Resulting pellets were resuspended in 10 volumes of SRB plus 1% Triton X-100 and subjected to 6 strokes in a Duall tissue homogenizer and centrifuged as above. Pellets were gently resuspended and centrifuged as above twice more in SRB including 1% (v/v) Triton X-100, twice in SRB lacking Triton X-100 and once in K-60 buffer (60 mM KCL, 20 mM MOPS, 2 mM MgCl₂ pH 7) before being resuspended in 5 volumes of K-60.

Detection of HNO modifications by Modified Biotin Switch. HNO modified thiols were detected using a modification to the standard biotin switch protocol [8]. In brief, 100 μg of rat myofibrils/treatment were diluted to 0.5 μg/μl in HEN (250 mM HEPES pH 7.7, 1 mM EDTA and 0.1 mM neocuproine) including 0.1% (w/v) SDS and exposed to a treatment for 10 min at 37° C. which was subsequently removed by acetone precipitation. Remaining free thiols were blocked by addition of 300 μl of HEN including 2.5% (w/v) SDS and 20 mM N-ethylmaleimide (NEM), incubated for 20 min at 50° C. Excess NEM was removed by acetone precipitation. HNO and/or NO modified thiols were reduced using 5 mM DTT or 1 mM ascorbate in 150 μl of HEN including 1% (w/v) SDS and biotinylated with 0.8 mM Biotin-HPDP (Pierce) for one hour at room temperature. Excess biotin-HPDP was removed by acetone precipitation (2 volumes) and resultant pellets were carefully washed with an additional volume of acetone. Biotinylated proteins were resuspended in 1 ml of HEN including 0.1% (w/v) SDS and captured by incubation with 15 μl of washed, packed Ultralink Immobilized Streptavidin (Pierce) for one hour at room temperature. Beads were washed four times in 50 bead volumes of HEN (twice including 0.1% (w/v) SDS, twice including 600 mM NaCl) and twice with EB (20 mM HEPES pH 7.7, 100 mM NaCl, 1 mM EDTA). Captured proteins were eluted with 40 μl of EB containing 100 mM DTT, mixed with 15 μl of 4×LDS sample buffer, boiled, separated by SDS PAGE and silver stained [9]. For MS studies, biotinylated proteins were digested overnight with trypsin (Promega) prior to capture and washed ten additional times with 5 mM ammonium bicarbonate/20% acetonitrile before being eluted in 100 μl of wash buffer including 100 mM DTT as described [10]. Captured peptides were identified using a LTQ linear ion trap tandem mass spectrometer (ThermoFinnigan, Waltham Mass. USA) with data searched against the rat IPI primary sequence database using the sorcerer searching platform (sagen).

Gel shift assay. 10 μg of rat myofibrils/treatment were diluted to 0.5 μg/μl in HEN including 0.1% (w/v) SDS and exposed to a treatment for 20 min at 37° C. Samples were diluted to 0.1 ug/ul in 1×LSD sample buffer, treated with 0, 5 or 100 mM DTT and separated by SDS PAGE. Proteins were silver stained or transferred to nitrocellulose and immunoblotted with primary antibodies for tropomyosin sarcomeric (CH1, Sigma-Aldrich Co., St. Louis, Mo., USA), actin (AC-40, Sigma-Aldrich Co., St. Louis, Mo., USA), myosin light chain 1 (MLM527, Abcam, Cambridge, Mass., USA) or myosin heavy chain (3-48, Abcam, Cambridge, Mass., USA). For some silver stained gel bands of interest in-gel digestion was done following the protocol outlined in Shevchenko et al. [9]. Gel slices of interest were excised from the gels cut into 1 mm³ pieces. Silver stained gel pieces were destained in 1:1(v/v) 30 mM Potassium ferricyanide and 100 mM Sodium thiosulfate and wash three times with ddH2O. Gel pieces were dehydrated in 100% acetonitrile and reswelled in 10 mM DTT and incubated at 55° C. for 1 hour. After the DTT solution was removed, a solution of 55 mM iodoacetimide was added and gel slices were incubated at room temperature protected from light. Gel slices were then washed 3 times with 50% (v/v) ACN, 25 mM (NH₄)HCO₃ and then fully dehydrated in 100% ACN and dried in a speed vac. Gel pieces were reswelled in a 12.5 ng/μL trypsin (Promega, Madison Wis. USA) solution containing 25 mM (NH₄)HCO₃ and incubated at 37° C. for >16 hours. Digested peptides were extracted by addition of 5% (v/v) formic acid and incubation for 15 min followed by addition of an equal volume of 100% ACN and 15 min incubation, this step was repeated and the extracts were combined. Proteins were identified using an Orbitrap LTQ tandem mass spectrometer (ThermoFinnigan, Waltham Mass. USA).

### Example 1

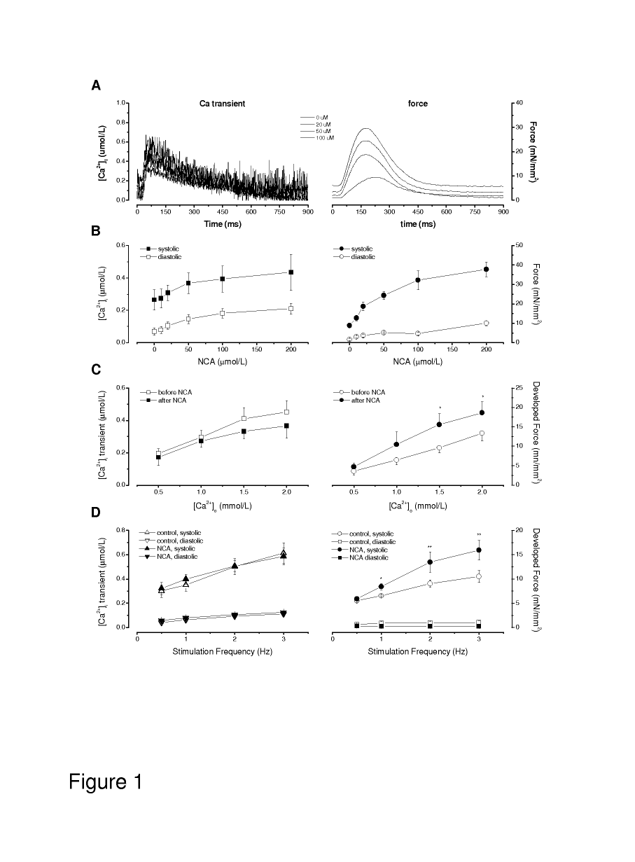

To isolate and characterize these effects two similar but distinct HNO donors were used. Angeli's Salt (AS) confers an increase in maximum force of contraction when applied to isolated trebecula or skinned muscle preparations without altering Ca₅₀ or the Hill coefficient. 1-nitrosocyclohexylacetate (NCA) is a new and mechanistically unique HNO donor that has been recently synthesized [11]. NCA releases HNO with minimal (<0.5%) NO and no nitrite at all, a known side product of AS decomposition and HNO release. When administered to isolated cardiac muscle, NCA increases force development in a dose dependent manner, from 20˜100 μmol/L, with no changes in diastolic force at 0.5 mmol/L [Ca²⁺]ₒ (FIG. 1 upper). NCA (100 μmol/L) increased force up to 32.3±4.8 mmHg/mm² (p<0.001 vs 8.8 mmHg/mm² in control muscles). Ca²⁺ transient did not rise significantly (0.39±0.08 vs. 0.27±0.06 μmol/L control, p=0.23) and diastolic Ca²⁺ increased only at high doses. Also, in the presence of NCA, systolic force remained significantly higher at any given external Ca²⁺. On the other hand, the amplitude of intracellular Ca²⁺ transients was not different from control (FIG. 1 middle). Force-frequency relationship was also enhanced by NCA without rising [Ca²⁺]ᵢ (FIG. 1 lower).

### Example 2

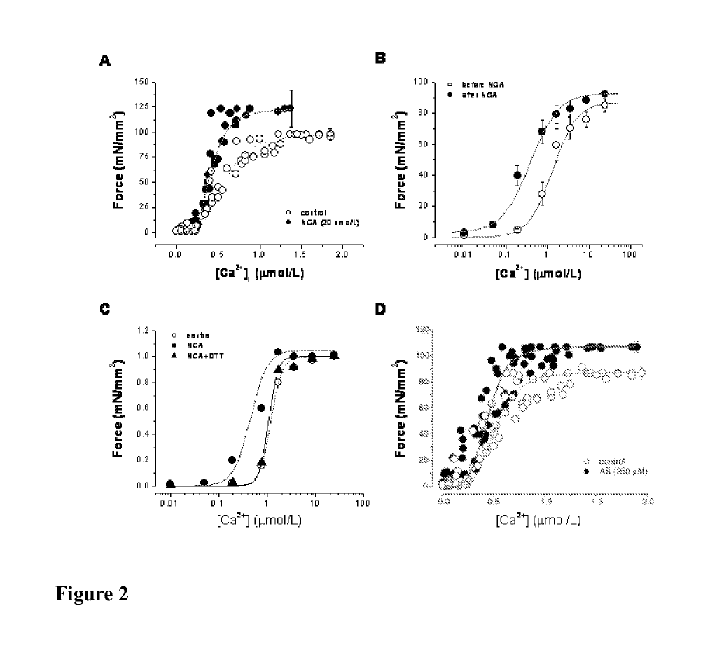

The above results show that force increased to a greater extent relative to Ca²⁺ transients, suggesting increased myofilament Ca²⁺ responsiveness by NCA. To further test this hypothesis, steady-state force-[Ca²⁺]ᵢ relations were obtained by tetanizing the muscles in the presence of ryanodine. The steady-state force-[Ca²⁺]ᵢ relations in control muscles and muscles exposed to NCA (20 μmol/L) are presented in FIG. 2A. Both maximal Ca²⁺-activated force (Fmax) and [Ca²⁺]ᵢ required for 50% of activation (Ca₅₀) increased significantly in muscles exposed to NCA (Fmax, 123±18 vs. 95±5 mN/mm², p<0.05; Ca₅₀, 0.42±0.01 vs. 0.57±0.03 mmol/L, p<0.004; Hill, 4.92±0.84 vs. 3.94±0.18, P=N.S.) Furthermore, the increased Ca²⁺ responsiveness persisted after skinning, indicating that NCA acts directly on the myofilaments (Fmax, 9411.6 vs. 8214.0 mN/mm², P=0.05; pCa₅₀, 0.30±0.13 vs. 1.35±0.36 μmol/L, P<0.001; Hill, 2.39±1.02 vs. 3.21±1.18, P=N.S) (FIG. 2B). Also, in skinned muscles, increases in myofilament Ca²⁺ sensitivity caused by HNO were completely abolished by DTT (FIG. 2C), thus confirming that HNO action is sensitive to reducing equivalents (3). Although HNO is the primary hydrolysis product of NCA (>50%), other potential products include acetic acid/sodium acetate and cyclohexanone. We have tested these compounds and they did not produce any appreciable effects in cardiac muscles (data not shown). For comparison, the steady state activation data for AS treated skinned muscle preparations is shown in FIG. 2D. Furthermore, 1-nitrosocyclohexyl pivalate, a compound of similar chemical structure but that does not release HNO had no effects (data not shown). Taken together, these data suggest the positive inotropic effect of NCA is specific to HNO.

### Example 3

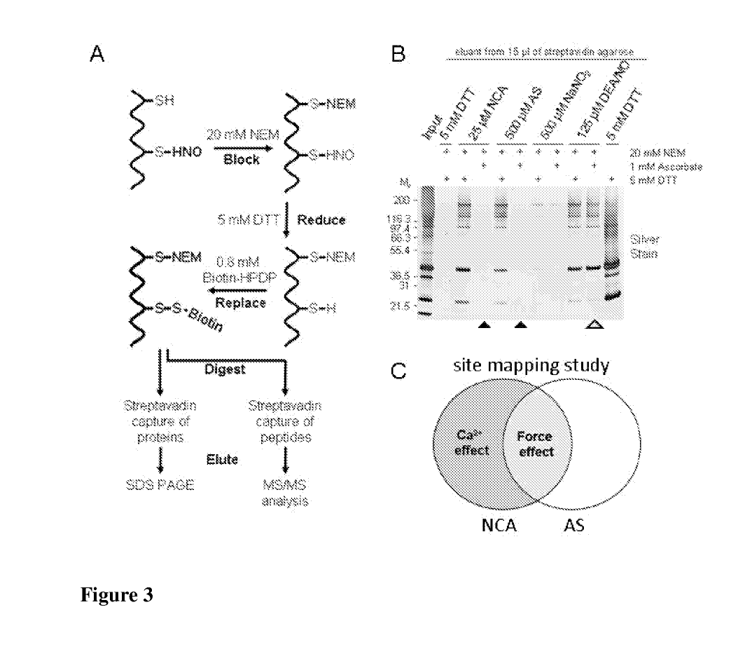

To investigate the modifications that underlie the difference seen in the functional effect of these two compounds we performed an analysis on the myofilament proteins, using a modified biotin-switch method, followed by mass spectrometry for identification (FIG. 3). Following treatment with HNO or control compounds, unmodified cysteines are blocked with NEM before treatment with a reducing agent and labeling the exposed cysteines with a biotin group (FIG. 3A). In this case, 5 mM DTT was used as the reducing agent because it was shown in the physiological studies to reverse the effect of the treatment. Streptavidin capture of intact proteins revealed that HNO modified proteins could be specifically captured and that HNO modifications were resistant to reduction with ascorbate, commonly used in biotin switch assays for S—NO groups (FIG. 3B). To determine the individual cysteines modified by HNO treatment the biotin switch assay was preformed on isolated cardiac myofibrils were treated with NCA (25 μM) or AS (500 μM) and compared to their decomposed/inactive equivalents. Tryptic or chymotryptic peptides containing the modified cysteine residues were captured and identified by LC/MS/MS. The experiments above involving HNO effects on cardiac myofilament proteins found that treatment with AS produced an increase in Fmax but had no effect on Ca₅₀ (3). To parse the role of candidate sites of modification between these two effects a comparative proteomics experiment was designed where modifications specific to NCA treatment would be considered candidates for the Ca²⁺ effect while sites identified in common between AS and NCA could be candidates for the force effect (FIG. 3C). A total of 8 proteins containing 12 potential sites of HNO modification were identified between the two treatments (Table 1). Of those, 3 proteins (tropomyosin Cys190, actin Cys257 and myosin heavy chain Cys947 and 1750) were found to be specifically modified by NCA. 7 sites were found to be in common between the two treatments.

TABLE 1
Sites of HNO modification detected by modified biotin switch assay.

| | | Treatment | | |
|---|---|---|---|---|
| | Peptide | Position of | | |
| Protein name | Identified | modified Cys | 25 μM NCA | 500 μM AS |
| tropomyosin (α) | CLELEEELKTVTNNLK ^{T} | 190 | 3^{a} | nd^{b} |
| | (SEQ ID NO: 1) | | | |
| α actin | DDEETTALVCDNGSLVK ^{T} | 12 | 3 | 3 |
| | (SEQ ID NO: 2) | | | |
| | VCDNGSLVKAGF ^{C} | 12 | nd | 2 |
| | (SEQ ID NO: 3) | | | |
| | LCYVALDFENEMATAASSSLEK ^{T} | 219 | 1 | 1 |
| | (SEQ ID NO: 4) | | | |
| | RCPETLFQPSF ^{C} | 257 | 1 | nd |
| | (SEQ ID NO: 5) | | | |
| myosin light chain 1 | ITYGQCGDVLR ^{T} | 81 | 2 | 2 |
| | (SEQ ID NO: 6) | | | |
| myosin heavy chain α | TECFVPDDKEEYVK ^{T} | 37 | 3 | 3 |
| | (SEQ ID NO: 7) | | | |
| | DIRTECFVPDDKEEY ^{C} | 37 | nd | 3 |
| | (SEQ ID NO: 8) | | | |
| α/β | ADAEERCDQL ^{C} | 907 | nd | 2 |
| | (SEQ ID NO: 9) | | | |
| | MDADLSQLQTEVEEAVQECR ^{T} | 1750 | 2 | nd |
| | (SEQ ID NO: 10) | | | |
| myosin heavy chain β | KLEDECELKR ^{T} | 947 | 3 | nd |
| | (SEQ ID NO: 11) | | | |
| α actinin | ELPPDQAQYCIKR ^{T} | 889 | 2 | 3 |
| | (SEQ ID NO: 12) | | | |
| myson binding protein C | VEFECEVSEEGAQVK ^{T} | 475 | 1 | nd |
| | (SEQ ID NO: 13) | | | |
| | ECEVSEEGAQVKW ^{C} | 475 | nd | 2 |
| | (SEQ ID NO: 14) | | | |
| troponin C (cardiac) | AAFDIFVLGAEDGCISTK ^{T} | 35 | 2 | 2 |
| | (SEQ ID NO: 15) | | | |

^{a}Value indicates number of independent observations of peptide
^{b}nd - not detected.
^{T} tryptic peptide, ^{C} chymotryptic peptide.

### Example 4

To map and evaluate the effects of HNO modification on individual Cys, a comparison was done between the changes induced by NCA to those of another HNO donor, Angeli's salt (AS). We have previously reported that AS increased Fₘₐₓ but did not affect Ca²⁺ sensitivity (Ca₅₀) in cardiac muscle. Using the modified biotin switch technique with different donors, a comparative proteomic strategy was devised to parse the effect of HNO: Cys modifications common to NCA and AS treatments were attributed to the increase in Fₘₐₓ while sites unique to NCA were considered candidates for the decrease in Ca₅₀. Biotin switch samples were digested overnight with trypsin or chymotrypsin; labeled peptides were captured with streptavidin and identified by LC/MS/MS. A total of 12 HNO-induced modified Cys on 8 proteins were identified between the two treatments, as shown in Table 2. Of those, 4 sites (TM Cys190, actin Cys257 and MHC Cys947 and Cys1750) were found to be uniquely induced by NCA.

TABLE 2
Sites of HNO modification determined by biotin switch assay.

| | | Position of |
|---|---|---|
| | Protein name | modified Cys |
| NCA | α-tropomyosin | 190 |
| | Actin | 257 |
| | myosin heavy chain | 947(β)* |
| | | 1750(α/β) |
| NCA/AS | myosin heavy chain | 37(α) |
| | myosin light chain 1 | 81 |
| | Actin | 12 |
| | | 219 |
| | α-Actinin | 889 |
| | myosin binding protein C | 475 |
| | troponin C | 35 |
| AS | myosin heavy chain | 907(α/β) |

*denotes sites present in different isoforms of myosin heavy chain sequence.

### Example 5

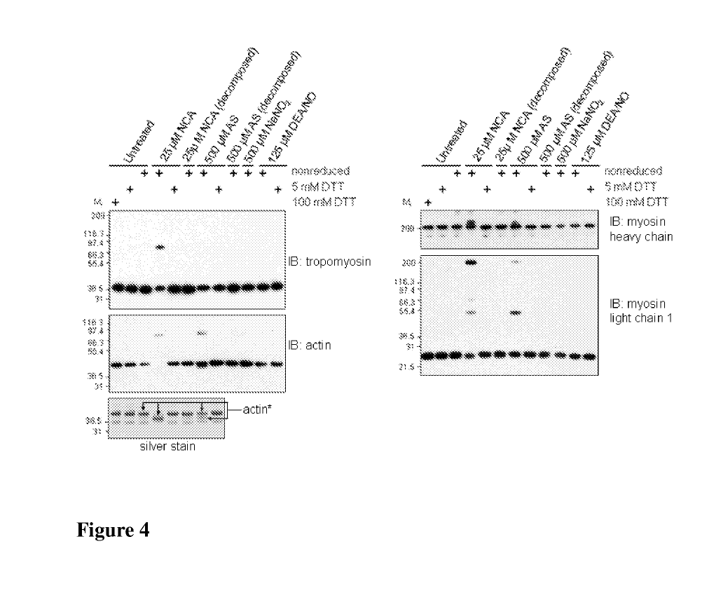

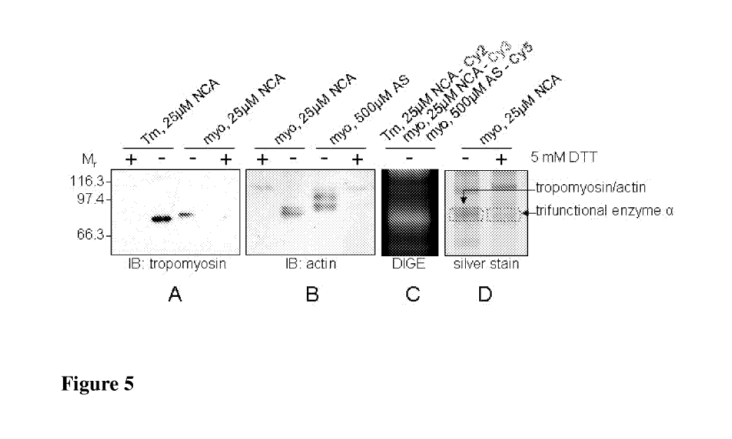

To confirm and characterize the candidate modifications, western blots from reducing/non-reducing 1D SDS-PAGE were performed in hopes of observing a molecular weight shift specific to HNO donor treatment. The analysis revealed higher molecular weight species for tropomyosin, actin, myosin heavy chain and myosin light chain 1, each of which were lost in the presence of 5 mM DTT (FIG. 4). Myosin heavy chain and light chain 1 were found to be modified in a similar manner with both NCA and AS treatment as predicted by the MS experiments, displaying two potential populations consistent with formation of an MLC1 homodimer (˜50 kDa) and an heavy—light chain heterodimer (>212 kDa). Tropomyosin, which contains only one Cys residue, was found to display a higher molecular weight species that was specific for NCA treatment, consistent with MS findings. Actin was found to possess higher molecular weight forms, at approximately 80 kDa in both NCA and AS treatment. Additionally, a loss of antibody epitope binding was observed for the monomeric form of actin with NCA treatment. MS analysis (LTQ or LTQ Orbitrap LC/MS/MS) of a silver stained gel bands revealed that with NCA that the actin monomer displays increased gel mobility. Analysis of other bands revealed that a similar, but less abundant, shift for actin in AS treated samples also occurred (FIG. 4, lower left image). Further analysis revealed that a molecular weight difference existed between the NCA and AS treated samples and that that the NCA induced tropomyosin band runs at the same apparent molecular weight as actin. This form ran higher than the dimer produced when purified TM was treated with NCA (FIGS. 5A and B). To evaluate this mobility difference more carefully a difference in gel electrophoresis (DIGE) experiment was performed where purified TM or myofibrils were treated, independently labeled with different fluorescent dyes, mixed and separated in a non-reducing gel (FIG. 5C). The analysis revealed a distinct myofibril NCA specific band (Cy3—green) higher than the purified tropomyosin homodimer (Cy2—blue) and below a series of AS specific bands (Cy5—red). MS analysis of the region identified actin and tropomyosin to be present at that location and lost under reducing conditions (FIG. 5D). These results indicate the presence of an actin-tropomyosin heterodimer and based on the specificity of the site mapping studies supports the conclusion that a disulphide bridge forms between Cys190 on TM and Cys257 on actin.

### Example 6

### In Skeletal Muscle, HNO Increases Ca²⁺ Sensitivity but not Maximum Force Production Due to the Lack of MHC-MLC1 Dimer Formation

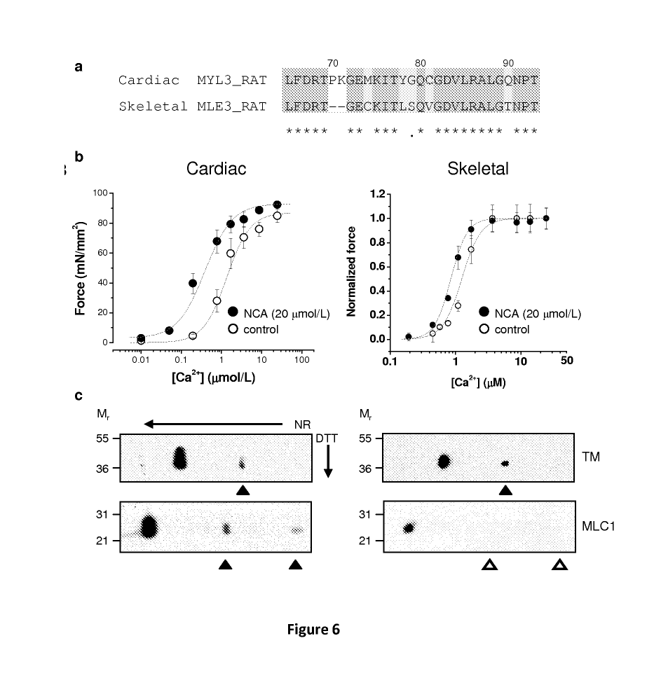

To determine if MLC1 Cys81 is involved in the increased maximum force production, the effect of HNO donors was investigated in skeletal muscle preparations. Skeletal muscle isoforms of myofilament proteins contain all of the potential target Cys except for MLC1, which lacks the candidate site Cys81 providing a natural mutant sequence (FIG. 6A). Steady-state force-[Ca²⁺]ᵢ relations of skeletal muscle before and after exposure to NCA (25 μmol/L) are presented in FIG. 6B. Ca₅₀ decreased significantly in the presence of NCA while Fₘₐₓ remained unchanged (Fₘₐₓ, 33±3.8 vs. 31.7±3.7 mN/mm², p=NS; Ca₅₀ 0.8±0.1 vs. 1.07±0.05 mmol/L, p<0.05; Hill, 4.36±0.81 vs. 3.47±0.82, p=NS). The same insensitivity of Fₘₐₓ to AS was also observed in skinned skeletal muscles (data not shown). 2D gel shift western blot analysis revealed an absence of higher molecular weight forms of MLC1 in NCA treated skeletal samples (FIG. 6C). These results indicate that MLC1 Cys81 is a critical residue and redox switch for the HNO induced increase in cardiac force production.

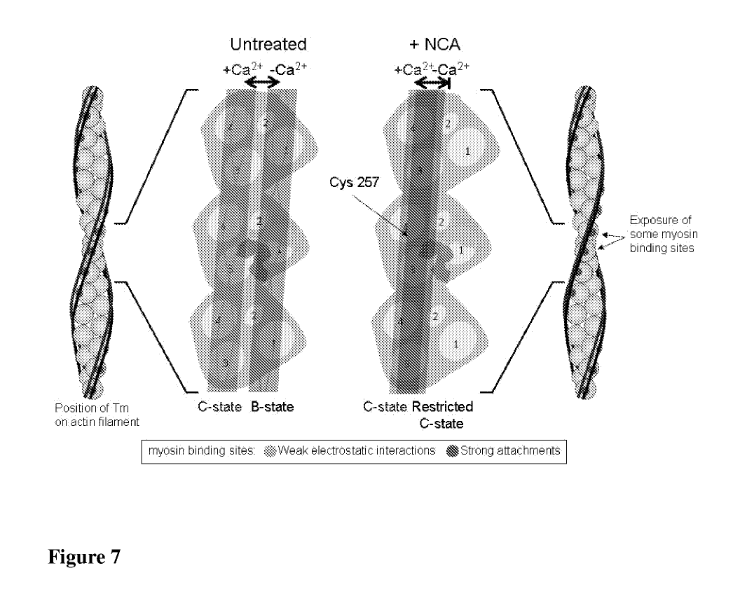

Although the inventors are not bound by any particular theory to explain the invention, they have developed two models to describe how the dimers identified would alter the maximum force of contraction and lower the requirement for calcium activation during contraction. First is the heterodimer formed between actin and tropomyosin which they have found increases calcium sensitivity. TM, a key regulatory protein in muscle contraction, determines the readiness of myofilaments for activation upon Ca²⁺ binding to TnC. During initiation of contraction, the 3 state model of steric hinderance suggests that Ca²⁺ binding to TnC releases TM allowing it to move over the surface of actin from the B (blocked) state where it covered subdomains 1 and 2 to the C (closed) state contacting subdomain 3 and 4 exposing the high affinity myosin binding sites (FIG. 7). We propose that crosslinking of tropomyosin (Cys 190) to actin (Cys 257, located in subdomain 4) restricts the movement of tropomyosin relative to actin shifting the average position to a greater C state character allowing greater access for myosin binding at lower Ca levels providing the mechanism for the enhanced response to Ca²⁺.

TM is a two-stranded (α-helical) coiled-coil dimer of two parallel 284-amino acid chains that wrap along the grooves of filamentous actin, spanning seven actin monomers. The secondary structure of TM is know to consist of 7 pseudo-repeats domains that mimic the structure of the actin filament and allow for coordinated association (12). Each repeat can be divided into and alpha (N-term) and beta (C-term) domain. Cys 190 of TM is located in the fifth peudo-repeat and would interact with the fifth of the seven actin monomers (13). The formation of disulphide bond via HNO chemistry requires that both thiol groups be in close proximity to each other. Previous investigation into the contacts made between actin and tropomyosin found that residues (167-184 alpha domain) came in direct contact with residues in actin subdomain 3 (14). This would alignment would be consistent with Cys 190 being in close association with Actin Cys 257 in subdomain 4. Additionally, in silico modeling predicted actin making points of contact with tropomyosin between Glu253 and Thr260 in subdomain 4 among others during calcium activation (15) further supporting the correct alignment and proximity for our proposed interaction.

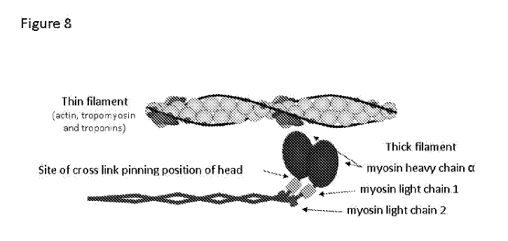

To address the change in maximum force seen in both the AS and NCA treated samples a mechanism has been proposed utilizing the observed interaction between myosin heavy chain and myosin light chain 1. The regulatory light chain 1 is positioned like a collar just below the head region of the heavy chain bringing the identified residues Cys 37 (MHC) and 81 (MLC1) in close proximity. The inventors propose that a slight restriction or pinning of the myosin head by light chain 1 could alter the angle at which the face contacts the binding sites on the thin filament. The tweaking of this interaction by a pinned head may allow for more contacts at high calcium levels increasing the max force generated. However the other candidates identified with both treatments make speculation on the eventual mechanism difficult. To address this they completed one additional experiment. Skeletal muscle is known to have some subtle isoform differences in the myofilament proteins. In particular the skeletal myosin light chain 1 lacks cysteine 81 from its sequence providing a natural mutagenesis situation. Steady state measurements of isolated skeletal muscle preparations treated with AS or NCA revealed the NCA specific decrease in Ca₅₀ but neither demonstrated the increase in maximum force (FIG. 8). This provides strong evidence for the effect of a pinned head interaction to drive the increase in maximal contractual force.

### CONCLUSIONS

Based on the analysis presented above, the inventors posit that any covalent crosslink, including disulfide bond formation that restricts the movement of TM or myosin heavy chain in this way would be a good candidate for producing the same functional effects of increasing calcium sensitivity and maximum force. As such, any bioactive compound with the potential to establish this type of interaction could be evaluated using the gel shift assay described in this work. Thus, detection of reagents which induce the formation of these dimers will also alter contractility. The dimers can be monitored at level of muscle or contractile tissue, isolated cells, isolated myofilbrils, isolated proteins or peptides of the regions where the crosslinking occurs. Detection of the crosslinking can be detected by functional change, formation of the covalent crosslink, antibodies or other detecting reagents, molecular weight alterations, mass spectroscopy based methods

While specific examples have been provided, the above description is illustrative and not restrictive. Any one or more of the features of the previously described embodiments can be combined in any manner with one or more features of any other embodiments in the present invention. Furthermore, many variations of the invention become apparent to those skilled in the art upon review of the specification. The scope of the invention should, therefore, be determined not with reference to the description herein, but instead should be determined with reference to the appended claims along with their full scope of equivalents.

References cited herein are listed below and are hereby incorporated by reference:
1. Gordon, A. M., Homsher, E., and Regnier, M. (2000) Regulation of contraction in striated muscle. Physiol Rev. 2, 853-924
2. Mudd, J. O., and Kass, D. A. (2008) Tackling heart failure in the twenty-first century. Nature. 7181, 919-28
3. Kass, D. A., and Solaro, R. J. (2006) Mechanisms and use of calcium-sensitizing agents in the failing heart. Circulation. 2, 305-15
4. Fukuto, J. M., Switzer, C. H., Miranda, K. M., and Wink, D. A. (2005) Nitroxyl (HNO): chemistry, biochemistry, and pharmacology. Annu Rev Pharmacol Toxicol. 335-55
5. Paolocci, N., Jackson, M. I., Lopez, B. E., Miranda, K., Tocchetti, C. G., Wink, D. A., Hobbs, A. J., and Fukuto, J. M. (2007) The pharmacology of nitroxyl (HNO) and its therapeutic potential: not just the Janus face of NO. Pharmacol Ther. 2, 442-58
6. Gao, W. D., Backx, P. H., Azan-Backx, M., and Marban, E. (1994) Myofilament Ca2+ sensitivity in intact versus skinned rat ventricular muscle. Circ Res. 3, 408-15
7. Murphy, A. M. & Solaro, R. J. Developmental difference in the stimulation of cardiac myofibrillar Mg2(+)-ATPase activity by calmidazolium. Pediatr. Res. 28, 46-49 (1990).
8. Jaffrey, S. R. & Snyder, S. H. The biotin switch method for the detection of S-nitrosylated proteins. Sci. STKE. 2001, 11 (2001).
9. Shevchenko, A., Wilm, M., Vorm, O., & Mann, M. Mass spectrometric sequencing of proteins silver-stained polyacrylamide gels. Anal. Chem. 68, 850-858 (1996).
10. Hao, G., Derakhshan, B., Shi, L., Campagne, F., & Gross, S. S. SNOSID, a proteomic method for identification of cysteine S-nitrosylation sites in complex protein mixtures. Proc. Natl. Acad. Sci. U.S. A 103, 1012-1017 (2006).
11. Sha, X., Isbell, T. S., Patel, R. P., Day, C. S., & King, S. B. Hydrolysis of acyloxy nitroso compounds yields nitroxyl (HNO). J. Am. Chem. Soc. 128, 9687-9692 (2006).
12. McLachlan, A. D., Stewart, M., & Smillie, L. B. Sequence repeats in alpha-tropomyosin. J. Mol. Biol. 98, 281-291 (1975).
13. McLachlan, A. D. & Stewart, M. The 14-fold periodicity in alpha-tropomyosin and the interaction with actin. J. Mol. Biol. 103, 271-298 (1976).
14. Lorenz, M., Poole, K. J., Popp, D., Rosenbaum, G., & Holmes, K. C. An atomic model of the unregulated thin filament obtained by X-ray fiber diffraction on oriented actin-tropomyosin gels. J. Mol. Biol. 246, 108-119 (1995).
15. Brown, J. H. et al. Structure of the mid-region of tropomyosin: bending and binding sites for actin. Proc. Natl. Acad. Sci. U.S. A 102, 18878-18883 (2005).

APPENDIX
Potential MRM transitions for the mass spec detection of Actin-Tropomyosin heterodimer

| protein | Candidate Cys | enzyme | m/z | peptide |
|---|---|---|---|---|
| actin | 257 | trypsin | 3846.785 | (R)CPETLFQPSFIGMESAGIHETTYNSIMKEDIDIR(K) |
| | | Chymo | 1324.635 | (F)RCPETLFQPSF(I) |
| | | double | 1168.534 | CPETLFQPSF |
| tropomyosin | 190 | trypsin | 1875.973 | (K)CLELEEELKTVTNNLK(S) |
| | | Chymo | 5975.081 | (Y)EEVARKLVIIESDLERAEERAELSEGKCLELEEELK |
| | | | | TVTNNLKSLEAQAEKY(S) |
| | | double | 1875.973 | (K)CLELEEELKTVTNNLK(S) |

Potential parent ions for disulfide bond linking Cys 190 and Cys 257 for MRM detection

| trypsin | double | Charge |
|---|---|---|
| 5720.758 | 3042.508 | 1 |
| 2860.879 | 1521.754 | 2 |
| 953.9596 | 507.5846 | 3 |
| 238.7399 | 127.1461 | 4 |

1. A method for screening for an agent that increases contractility in a contractile cell comprising the steps of:
a. contracting a test agent with a composition comprising contractile proteins from said cell; and
b. measuring the formation of at least one cross link to form a heterodimer between said contractile proteins,
wherein the formation of at least one cross link between said proteins is indicative of an potential agent for increasing contractility of said cell.

2. The method of claim 1 wherein the contractile cell is a muscle cell.

3. The method of claim 2 wherein the muscle cell is a smooth muscle cell, a skeletal muscle cell or a cardiac muscle cell.

4. The method of claim 1 wherein the contractile cell is a cell having motility.

5. The method of claim 4 wherein the cell is a blood cell.

6. The method of claim 1 wherein the contractile proteins are tropomyosin (TM) and actin, myosin heavy and myosin light chains.

7. The method of claim 1 wherein the link is a disulfide bond.

8. The method of claim 7 wherein the link is formed between Cysteine residue 190 of TM and Cys 257 of actin, or between Cys 37 of myosin heavy chain and Cys 81 of myosin light chain 1.

9. The method of claim 1 wherein cross-linkage is measured by a method selected from the group consisting of molecular weight assay, antibody assay, molecular sieving assay, and mass spectrometry.

10. A method for screening for an agent that reduces contractility in a contractile cell comprising the steps of:
a. contracting a test agent with a composition comprising contractile proteins from said cell in heterodimer form; and
b. measuring the disruption of at least one cross link between said contractile proteins; wherein the disruption of at least one cross link between said proteins is indicative of a potential agent for decreasing contractility of said cell.

11. The method of claim 10 wherein the contractile cell is a muscle cell.

12. The method of claim 11 wherein the muscle cell is a smooth muscle cell, a skeletal muscle cell or a cardiac muscle cell.

13. The method of claim 10 wherein the contractile proteins are selected from TM and actin, and myosin heavy and myosin light chains.

14. The method of claim 10 wherein the link is a disulfide bond.

15. The method of claim 14 wherein the link is formed between Cysteine residue 190 of TM and Cys 257 of actin, or between Cys 37 of myosin heavy chain and Cys 81 of myosin light chain 1.

16. The method of claim 10 wherein cross-linkage is measured by a method selected from the group consisting of molecular weight assay, antibody assay, molecular sieving assay, and mass spectrometry.

17. A diagnostic method comprising the step of detecting and/or measuring the level of a heterodimer comprised of contractile proteins in a biological sample wherein the presence and/or level of said heterodimer is correlated with a diagnosis, prognosis or treatment outcome.

18. The method of claim 17 wherein the diagnosis, prognosis or treatment outcome is for a cardiac disease or disorder.

19. The method of claim 18 wherein the cardiac disease or disorder is heart failure or myocardial stunning.

20. The method of claim 17 wherein the diagnosis, prognosis or treatment outcome is for a disease or disorder of skeletal muscle.

21. The method of claim 20 wherein the disease or disorder of skeletal muscle is muscle cramping.

22. The method of claim 17 wherein the diagnosis, prognosis or treatment outcome is for a disease or disorder of smooth muscle.

23. The method of claim 22 wherein the disease or disorder of smooth muscle is selected from the group consisting of irritable bowl and gastric mobility, asthma, vascular spasm, uterine contraction involved in premature delivery of delivery itself, and menstrual cramps.

24. The method of claim 17 wherein the diagnosis, prognosis or treatment outcome is for a cancer.

25. The method of claim 17 wherein the biological sample is a blood sample, a tissue biopsy or a bodily fluid.

26. The method of claim 25 wherein the biological sample is a serum or plasma sample.

27. A kit for screening for an agent that modifies cellular contractility, said kit comprising at least one antibody directed to a contractile protein, a denaturing gel, and a control sample comprising a contractile protein in homodimeric form.

28. The kit of claim 27, additionally comprising a nitrocellulose membrane.

29. The kit of claim 27, additionally comprising at least one digestive enzyme.

30. The kit of claim 29, wherein the digestive enzyme is trypsin or chymotrypsin.

31. An isolated biomarker comprising a heterodimer comprised of contractile proteins, wherein the presence of said biomarker in a biological sample is indicative of altered contractility of a contractile cell.

32. An isolated biomarker consisting of cross-linked contractile proteins having a molecular weight which indicate the presence of a heterodimer.
